(19) United States
(12) Patent Application Publication
(10) Pub. No.: US 2022/0338878 A1
(43) Pub. Date: Oct. 27, 2022

(54) MECHANICAL DETACHMENT SYSTEM WITH A LEVER STRUCTURE FOR DEPLOYMENT OF ENDOVASCULAR DEVICES

(71) Applicant: DeepIn Technologies, LLC, San Jose, CA (US)

(72) Inventors: Ross Soltanian, San Jose, CA (US); Gregory M. Mast, Morgan Hill, CA (US); Thomas Edmund Kiernan, Fremont, CA (US)

(21) Appl. No.: 17/722,162

(22) Filed: Apr. 15, 2022

Related U.S. Application Data
(60) Provisional application No. 63/178,858, filed on Apr. 23, 2021.

(51) Int. Cl. A61B 17/12

(52) U.S. Cl. CPC A61B 17/12145; A61B 17/12031; A61B 17/12113; A61B 2017/12095

(57) ABSTRACT

An endovascular system employs a lever structure to deploy an embolic device at a target site in the vasculature of a patient. The endovascular system comprises an elongate tubular member provided with a lever structure and an elongate detachment wire to assert an effort to the proximal portion of a lever member outwardly, thereby generating a load to the distal portion of the lever member inwardly to allow the lever structure to engage and secure the embolic device. The elongate detachment wire is disengageable from the lever structure to remove the effort asserted to the proximal portion of the lever member, thereby removing the load off the distal portion of the lever member to allow the lever structure to disengage and release the embolic device.

### CROSS-REFERENCE TO RELATED APPLICATIONS

This application claims priority to U.S. provisional patent application No. 63/178,858 filed Apr. 23, 2021 entitled “Mechanical Detachment System for Deployment of Endovascular Devices,” the disclosure of which is hereby incorporated by reference in its entirety.

### TECHNICAL FIELD

This application relates generally to medical devices and methods. In particular, various embodiments of an endovascular system and a mechanical detachment system for deploying implants within the vasculature of a human body are described.

### BACKGROUND

Implants such as embolic devices are known in treatment of vascular disorders such as aneurysms. An aneurysm is a bulge or swelling formed on a wall of an artery in the brain or other locations of a human body. A brain aneurysm can cause severe pain, and if ruptured, lead to fetal stroke. In a non-invasive or minimally invasive treatment of aneurysms, an embolic device such as a coil, stent, or intrasaccular web may be placed in or at the aneurysm to isolate the aneurysm from blood flow, and/or, promote thrombus formation at the site. The placement of an embolic device is typically accomplished using a delivery system, which steers the embolic device through the vasculature of the patient to the location of the aneurysm. Once positioned at or in the aneurysm, the embolic device is detached from the delivery system by applying thermal or electrolytic power or by activating a mechanical detachment mechanism.

Conventional systems or methods for delivering and deploying embolic devices often present risks of prematurely or inadvertently releasing the embolic devices before deployment at the target location. This is especially the case in treating brain aneurysms during which a delivery system would have to navigate through a tortuous vascular path, where advancement and retraction of the delivery system is often required in order to accurately place the embolic device to reduce errors that may result in significant damage to the brain.

Therefore, there remains a general need for improved systems and methods of delivering implants for treating vascular disorders such as brain aneurysms. It would be desirable to provide a delivery system that can reliably and controllably navigate through the vasculature of a human body in delivering implants and reduce the risks of premature or inadvertent release of the implants before deployment at a target site.

### SUMMARY

In one aspect, embodiments of the disclosure feature a detachment system for deploying an implantable device in a patient. In general, an embodiment of the detachment system comprises a tubular member provided with a lever structure at the distal end portion of the tubular member configured to secure and release the implantable device, and a detachment wire configured to control the lever structure. The lever structure includes a fulcrum and a lever member pivotable about the fulcrum. The detachment wire is configured to engage the lever structure to assert an effort to the proximal portion of the lever member outwardly, thereby generating a load to the distal portion of the lever member inwardly to allow the lever structure to engage and secure the implantable device. The detachment wire is disengageable from the lever structure to remove the effort asserted to the proximal portion of the lever member, thereby removing the load off the distal portion of the lever member to allow the lever structure to disengage and release the implantable device.

In various embodiments of the aspect, the lever member and the fulcrum of the lever structure can be formed by cutting a tubular wall. In a specific embodiment, the tubular wall has a first pair of opposing cut-outs distal of a segment of the tubular wall, a second pair of opposing cut-outs proximal of the segment, and an arc-shaped cut-out across the second pair of opposing cut-outs, forming the lever member pivotable about the segment.

In various embodiments of the aspect, the lever member can be formed to provide a configuration of the lever structure wherein when the lever structure is engaged with the detachment wire and the implantable device, the central longitudinal axis of the lever structure is generally concentric with the central longitudinal axis of the tubular member.

In various embodiments of the aspect, the lever member is formed to provide a configuration of the lever structure wherein when the lever structure is disengaged from the detachment wire and the implantable device, the lever member is oriented outwardly distally.

In various embodiments of the aspect, the distal end portion of the detachment wire has a cylindrically shaped surface dimensioned to engage the inner surface of the lever member to assert the effort to the proximal portion of the lever member outwardly.

In various embodiments of the aspect, the distal portion of the lever structure comprises a step feature configured to facilitate retainment of the implantable device.

In various embodiments of the aspect, the proximal end portion of the elongate tubular member comprises a tip section that is separatable from the tubular member to allow a user to pull the detachment wire.

In another aspect, embodiments of the disclosure feature an endovascular system. In general, an embodiment of the endovascular system comprises an embolic device and a delivery device operable to deploy the embolic device at a target site in the vasculature of a patient. The delivery device comprises an elongate tubular member provided with a lever structure at the distal end portion of the elongate tubular member and an elongate detachment wire configured to control the lever structure. The lever structure comprises a fulcrum and a lever member pivotable about the fulcrum. The elongate detachment wire is configured to engage the lever structure to assert an effort to the proximal portion of the lever member outwardly, thereby generating a load to the distal portion of the lever member inwardly to allow the lever structure to engage and secure the embolic device. The elongate detachment wire is disengageable from the lever structure to remove the effort asserted to the proximal portion of the lever member, thereby removing the load off the distal portion of the lever member to allow the lever structure to disengage and release the embolic device.

In various embodiments of the aspect, the lever member and the fulcrum of the lever structure can be formed by cutting a tubular wall. In a specific embodiment, the tubular wall has a first pair of opposing cut-outs distal of a segment of the tubular wall, a second pair of opposing cut-outs proximal of the segment, and an arc-shaped cut-out across the second pair of opposing cut-outs, forming the lever member pivotable about the segment.

In various embodiments of the aspect, the lever member can be formed to provide a configuration of the lever structure wherein when the lever structure is engaged with the detachment wire and the implantable device, the central longitudinal axis of the lever structure is generally concentric with the central longitudinal axis of the tubular member.

In various embodiments of the aspect, the lever member is formed to provide a configuration of the lever structure wherein when the lever structure is disengaged from the detachment wire and the implantable device, the lever member is oriented outwardly distally.

In various embodiments of the aspect, the distal end portion of the detachment wire has a cylindrically shaped surface dimensioned to engage the inner surface of the lever member to assert the effort to the proximal portion of the lever member outwardly.

In various embodiments of the aspect, the embolic device comprises a coupling member having a proximal end portion configured to engage the inner surface of the lever member when the load is generated to the distal portion of the lever member inwardly to secure the embolic device.

In various embodiments of the aspect, the proximal end portion of the coupling member of the embolic device has an enlarged dimension generally in a spherical shape. The proximal end portion of the coupling member is pivotable when engaged with the inner surface of the lever member, thereby allowing the embolic device to pivot when secured by the lever structure. In a specific embodiment, the distal portion of the lever structure comprises a step feature configured to facilitate retainment of the embolic device.

In various embodiments of the aspect, the proximal end portion of the elongate tubular member comprises a tip section that is separatable from the elongate tubular member to allow a user to pull the detachment wire.

In various embodiments of the aspect, the embolic device comprises an embolic coil, a stent, or an intrasaccular device.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a brief summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1A is an isometric view of an example of an endovascular system, showing an embolic device and a delivery device in a delivery configuration according to embodiments of the disclosure.

FIG. 1B is a plan view of the example endovascular system shown in FIG. 1A according to embodiments of the disclosure.

FIG. 1C is a cross-sectional view of the example endovascular system shown in FIG. 1B, taken along line A-A.

FIG. 2A is an isometric view of an example of an endovascular system, showing an embolic device and a delivery device in a release configuration according to embodiments of the disclosure.

FIG. 2B is a plan view of the example endovascular system shown in FIG. 2A according to embodiments of the disclosure.

FIG. 2C is a cross-sectional view of the example endovascular system shown in FIG. 2B, taken along line B-B.

FIG. 3 depicts an example of a detachment wire according to embodiments of the disclosure.

FIG. 4A is an isometric view and FIG. 4B an end view of an example of a lever structure according to an embodiment of the disclosure.

FIG. 5A is an isometric view and FIG. 5B an end view of an example of a lever structure according to another embodiment of the disclosure.

FIG. 6A is an isometric view and FIG. 6B an end view of an example of a lever structure according to a further embodiment of the disclosure.

FIG. 7A is an isometric view and FIG. 7B an end view of an example of a lever structure according to a further embodiment of the disclosure.

FIG. 8A is an isometric view and FIG. 8B an end view of an example of a lever structure according to a further embodiment of the disclosure.

FIG. 9A is an isometric view and FIG. 9B an end view of an example of a lever structure according to a further embodiment of the disclosure.

FIG. 10A is an isometric view and FIG. 10B a cross-sectional view of an example of an endovascular system in a delivery configuration according to some embodiments of the disclosure.

FIG. 11A is an isometric view and FIG. 11B a cross-sectional view of the example endovascular system shown in FIGS. 10A-10B in a release configuration according to some embodiments of the disclosure.

### DETAILED DESCRIPTION OF EMBODIMENTS

With reference to the figures, various embodiments of an endovascular system and a detachment system for delivering and deploying implants will now be described. It should be noted that the figures are intended for illustration of embodiments but not for exhaustive description or limitation on the scope of the disclosure. Alternative structures and components will be readily recognized as being viable without departing from the principle of the claimed invention.

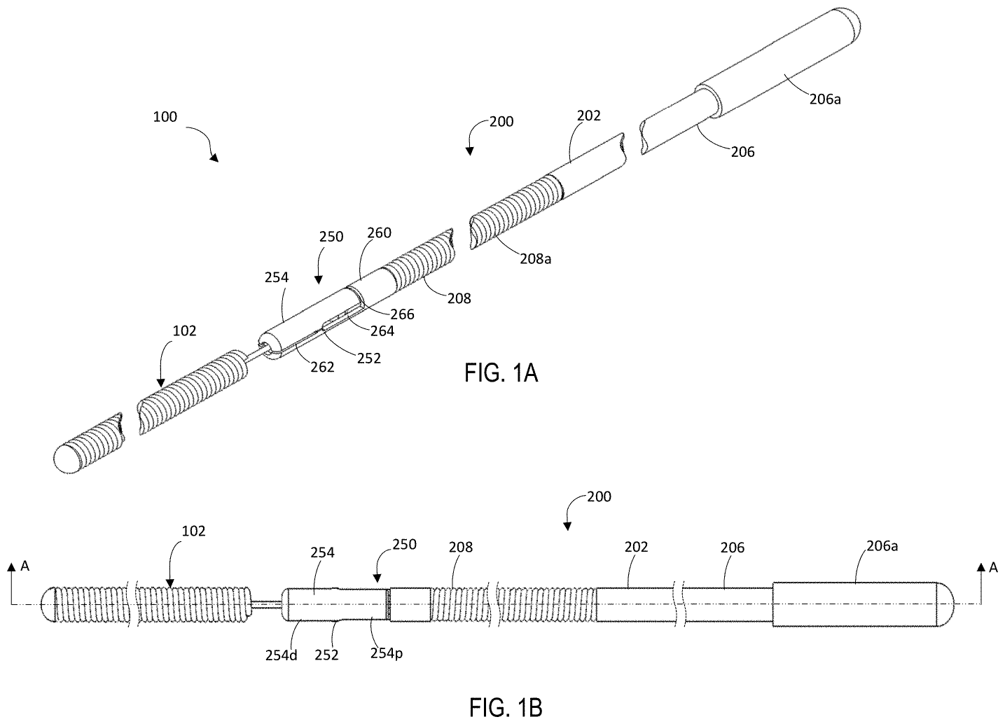

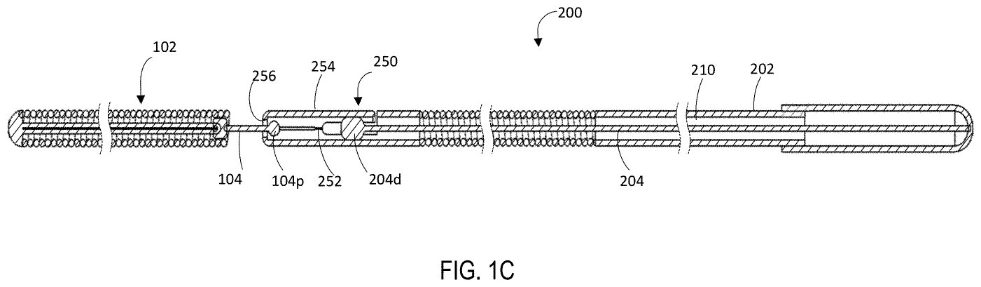

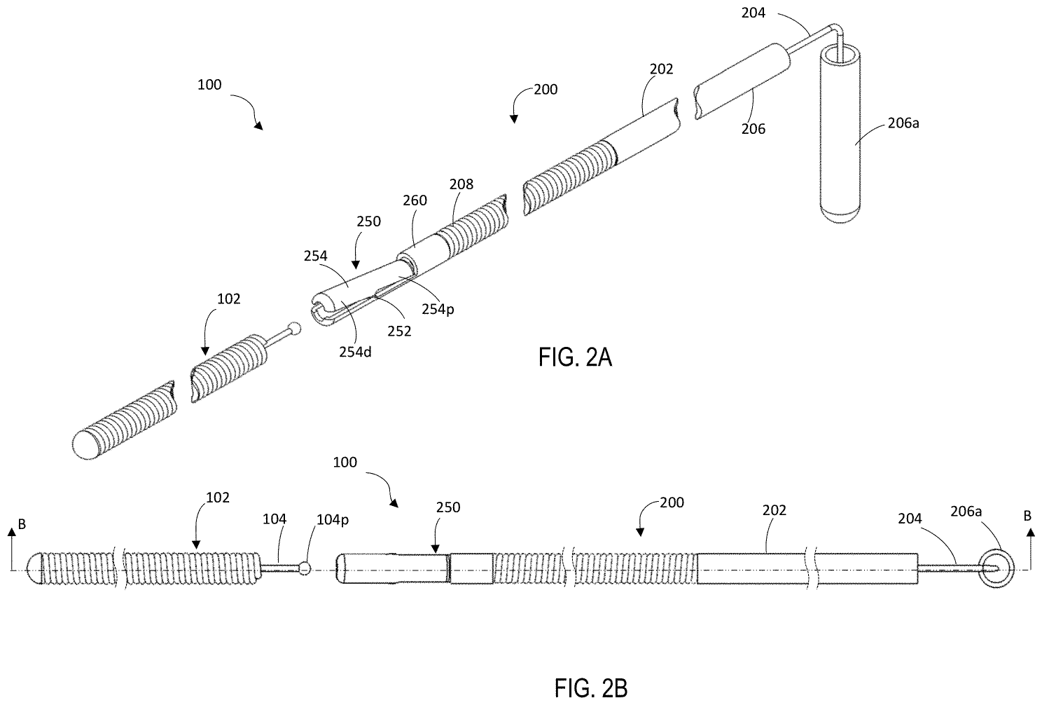

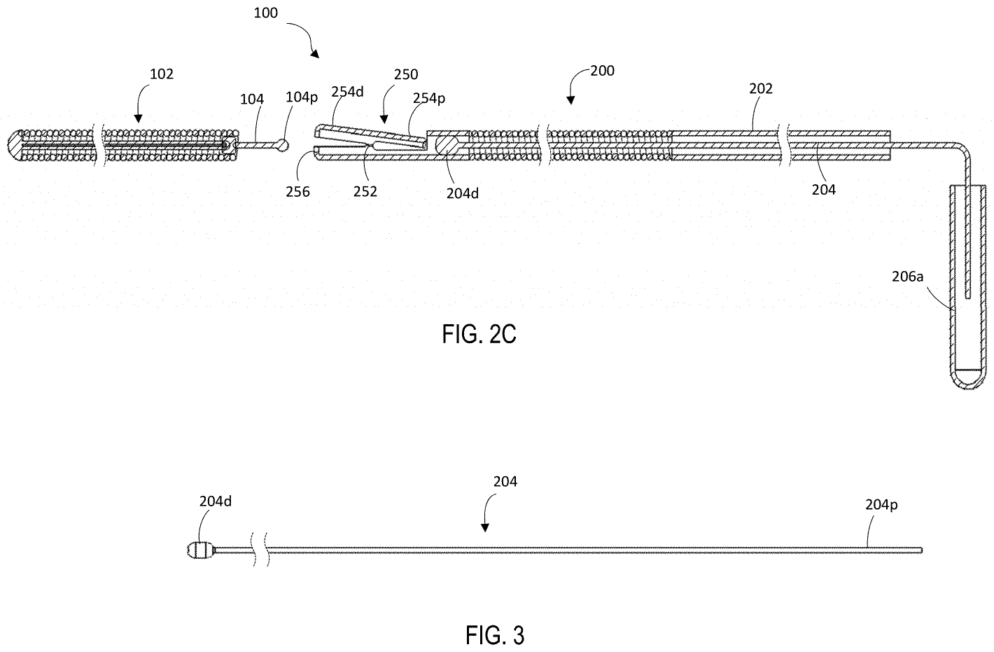

FIGS. 1A-1C and 2A-2C illustrate an example of an endovascular system 100 according to one embodiment of the disclosure. In a broad overview, the example endovascular system 100 includes an implantable device 102 and a delivery device 200 operable to deliver and deploy the implantable device 102 at a target site in a patient. The delivery device 200 generally includes an elongate tubular member 202 and an elongate detachment wire 204 (FIG. 1C). A lever structure 250 is provided at the distal end portion of the elongate tubular member 202 to retain or detach the implantable device 102. In a delivery configuration, as depicted in FIGS. 1A-1C, the lever structure 250 is engaged with the detachment wire 204, allowing the lever structure 250 to retain or secure the implantable device 102 for advancement and/or retraction in the vasculature of a patient. To deploy the implantable device 102 at a target site, the detachment wire 204 is disengaged from the lever structure 250, allowing the lever structure 250 to release the implantable device 102, as depicted in FIGS. 2A-2C.

With reference to FIGS. 1A-1C, the elongate tubular member 202 of the delivery device 200 includes a proximal end portion 206, a distal end portion 208, and defines a lumen 210 (FIG. 1C). The proximal end portion 206, or a portion of the proximal end portion 206, may remain outside of the patient and is accessible to the user or physician when the endovascular system 100 is in use. The distal end portion 208 may be sized and dimensioned to reach a remote location in the vasculature of the patient, such as in a blood vessel adjacent to an aneurysm neck, a bifurcated blood vessel, an occlusion in a blood vessel, or the like.

With reference to FIGS. 1A-1C, the elongate tubular member 202 may include one or more sections or regions along its length. Each of the one or more sections or regions may have different configurations and/or characteristics. For example, the distal end portion 208 of the elongate tubular member 202 may include a flexible section or region 208a constructed e.g., of a coil to provide proper bending or deflection. A flexible distal end portion 208a would allow the endovascular system 100 to navigate more easily through tortuous regions of the vasculature to remote locations in the patient. The proximal end portion 206 may be constructed of a stiffer material e.g., of a rigid metal hypertube, to provide structural stability and sufficient pushability. Therefore, according to embodiments of the disclosure, the proximal end portion 206 of the elongate tubular member 202 may serve as a pusher to advance or retract the implantable device 102 coupled to the distal end portion 208 of the elongate tubular member 202. In general, the elongate tubular member 202 or a section of the elongate tubular member 202 may be constructed from suitable metals such as stainless steel, nickel, titanium, nitinol, alloys of metals, biocompatible polymers, shape memory polymers, or combinations thereof. The distal end portion 208 of the elongate tubular member 202 may have an outer diameter less than the outer diameter of the proximal end portion 206 to reduce the profile of the distal end portion 208 and facilitate navigation through tortuous vasculature. By way of example, the outer diameter of the elongate tubular member at the distal end portion 208 may range from about 0.012 inch to about 0.027 inch. The outer diameter of the elongate tubular member 202 at the proximal end portion 206 may range from about 0.012 inch to about 0.027 inch. The total length of the elongate tubular member 202 may range from about 90 cm to about 130 cm. While not shown, the elongate tubular member 202 may include one or more markers which can be viewed e.g., via fluoroscopy, to assist the physician in operation of the endovascular system 100.

With reference to FIGS. 1A-1C and 2A-2C, the elongate tubular member 202 may comprise a proximal tip section 206a that can break or otherwise be separated from the elongate tubular member 202 for operation of the lever structure 250 (FIGS. 2A-2C). The breakable or separatable proximal tip section 206a allows the physician to access the detachment wire 204 once the implantable device 102 is properly positioned at the target site. As such, the physician may pull the attachment wire 204 at the proximal end of the endovascular system 100 to disengage the attachment wire 204 from the lever structure 250 located at the distal end of the endovascular system 100. The disengagement of the detachment wire 204 from the lever structure 250 allows the implantable device 102 to be released from the lever structure 250, to be described in greater detail below. The proximal tip section 206a can be coupled to the elongate tubular member 202 via soldering, welding, adhesive bonding, or other suitable means during manufacturing of the endovascular system 100, which can break e.g., upon bending by the physician during operation.

With reference to FIGS. 1A-1C and 2A-2C, the elongate tubular member 202 comprises a lever structure 250 at the distal end portion 208 of the tubular member 202. The lever structure 250 may serve as a retainment and detachment mechanism of the endovascular system 100. During delivery, the lever structure 250 can retain or secure the implantable device 102 to navigate through the vasculature of a patient. Once properly positioned at a target site, the lever structure 250 can controllably release or deploy the implantable device 102. As illustrated, the lever structure 250 generally comprises a fulcrum 252 and a lever member 254 pivotable about the fulcrum 252. The lever member 254 comprises a proximal portion 254p proximal of the fulcrum 252 and a distal portion 254d distal of the fulcrum 252 (FIGS. 1B, 2A, and 2C). When the detachment wire 204 is engaged with the the lever structure 250, as depicted in FIGS. 1A-1C, the detachment wire 204 may assert an effort to the proximal portion 254p of the lever member 254 upwardly or outwardly, thereby generating a load to the distal portion 254d of the lever member 254 downwardly or inwardly to allow the lever structure 250 to engage and secure the implantable device 102. The detachment wire 204 is disengageable from the lever structure 250, e.g., by being pulled proximally, to remove the effort asserted to the proximal portion 254p of the lever member 254, thereby removing the load off the distal portion 254d of the lever member 254 to allow the lever structure 250 to disengage and release the implantable device 102, as shown in FIGS. 2A-2C. As used herein, the term “outwardly” and its grammatical equivalent refers to a direction or an orientation that is away from the elongate tubular member. The term “inwardly” or its grammatical equivalent refers to a direction or an orientation that is towards the elongate tubular member.

With reference to FIGS. 1A-1C and 2A-2C, the lever structure 250 may be formed by cutting a tube or tubular wall 260 (FIG. 1A). By way of example, a tubular wall 260 may be cut to have a first pair of opposing cut-outs 262 distal of a segment 252 of the tubular wall 260, a second pair of opposing cut-outs 264 proximal of the segment 252, and an arc-shaped cut-out 266 across the second pair of opposing cut-outs 264, forming the lever member 254 pivotable about the segment or fulcrum 252 (FIG. 1A). Any suitable technology can be used to cut the tubular wall 260, including laser cutting, etching, etc. known in the art. It should be noted that the tubular wall 260 can be a separate piece of component and the lever structure 250 can be formed by cutting the separate piece of component and then coupled to the distal end portion 208 of the elongate tubular member 202 e.g., via soldering, welding, adhesive bonding or other suitable means. Alternatively, the tubular wall 260 can be an integral portion of an elongate tubular member 202 and the lever structure 250 can be formed by cutting the tubular wall portion. The tubular wall 260 can be constructed of a material comprising nitinol, stainless steel, or other metals or metal alloys, or a biocompatible polymeric material.

With reference to FIGS. 1A-1C, in a preferred embodiment, the lever structure 250 can be formed such that when the lever structure 250 is in a delivery configuration, or when the lever structure 250 is engaged with the detachment wire 204, a central longitudinal axis of the lever structure 250 is generally concentric with a central longitudinal axis of the tubular member 202. The concentric configuration can advantageously reduce the risks of inadvertent or premature release of the implantable device 102 when the endovascular system 100 is advanced or retracted in navigating through a tortuous vascular path in the patient.

With reference to FIGS. 2A-2C, in a preferred embodiment, the lever structure 250 can be formed such that when the lever structure 250 is in a release or natural configuration, or when the lever structure 250 is disengaged from the detachment wire 204, the lever member 254 is oriented upwardly distally (FIG. 2A and 2C). This may be accomplished by using a memory material and/or shaping the cut-outs in the tubular wall 260. By way of example, the opposing cut-outs 264 proximal of the fulcrum 252 may be wider than the opposing cut-outs 262 distal of the fulcrum 252 (FIG. 1A), and a memory material comprising nitinol may be used for making the lever structure 250.

With reference to FIG. 3, 1A-1C and 2A-2C, the elongate detachment wire 204 has a proximal end portion 204p and a distal end portion 204d. The distal end portion 204d of the detachment wire 204 is configured to engage the lever structure 250, allowing the lever structure 250 to retain or secure the implantable device 102 for delivery. The distal end portion 204d of the elongate detachment wire 204 is disengageable from the lever structure 250, e.g., by pulling the detachment wire 204 proximally, allowing the lever structure 250 to release the implantable device 102 for deployment at the target site. The proximal end portion 204p of the detachment wire 204 may be coupled to the proximal tip of the tubular member 202 e.g., by soldering, welding, adhesive bonding, or other suitable means. The detachment wire 204 can be made of a material comprising a metal such as stainless steel, or metal alloy.

With reference to FIG. 3, 1A-1C and 2A-2C, in some embodiments, the distal end portion 204d of the elongate detachment wire 204 may have an enlarged dimension configured to engage the lever structure 250. The enlarged distal end portion 204d of the detachment wire 204 may be sized to fit against the inner surface of the lever member 254 to outwardly assert an effort to the lever member 254 (FIG. 1C). In some embodiments, the distal end portion 204d of the detachment wire 204 may have a curved surface such as a cylindrical surface to enhance stability and facilitate engagement with or disengagement from the lever structure 250. By way of example, the lever member 250 may be formed by cutting a tubular wall and thus have a curved inner surface. A cylindrical surface of the distal end portion 204d of the detachment wire 204 can facilitate engagement or disengagement of the detachment wire 204 with the curved inner surface of the lever member 254. Alternatively, the distal end portion 204d of the detachment wire 204 may have other shapes such as a spherical, conical, or cubical shape. By way of example, the enlarged distal end 204d of the detachment wire 204 may have a cross-sectional diameter ranging from about 0.002 inch to about 0.006 inch. The proximal end portion 204p of the detachment wire 204 may have a cross-sectional diameter ranging from about 0.004 inch to 0.012 inch.

With reference to FIGS. 1A-1C and 2A-2C, the implantable device 102 can be any suitable implant compatible with the delivery system 100 of the disclosure. By way of example, the implantable device 102 can be an embolic device such as a coil, stent, or intrasaccular device used for treatment of brain aneurysms. The implantable device 102 can also be a dilation device, filter, thrombectomy device, atherectomy device, flow restoration device, etc. for treatment of other disorders in other locations of a human body. For illustration purpose, the implantable device is shown as an embolic coil in FIGS. 1A-1C and 2A-2C. It should be noted that the scope of the disclosure and appended claims are not limited to a specific type of an implantable device, and the retainment and detachment mechanism disclosed herein can be used with any other suitable implantable devices.

With reference to FIGS. 1A-1C and 2A-2C, the implantable device 102 may comprise a coupling member 104 for connecting the implantable device 102 with the lever structure 250. The coupling member 104 may have a distal portion attached or fixedly attached to the implantable device, and a proximal end 104p sized and shaped or configured to facilitate engagement and disengagement of the implantable device 102 with the lever structure 250 (FIGS. 1C and 2A-2C). By way of example, the lever member 254 may be formed by cutting a tubular wall 260 and thus have a curved inner surface. The proximal end 104p of the coupling member 104 may have a spherical surface to facilitate engagement or disengagement of the implantable device 102 with the curved inner surface of the lever member 254. In a specific embodiment, the proximal end 104p of the coupling member 104 may have a ball or spherical shape. The ball or spherical shape of the proximal end 104p of the coupling member 104 allows the implantable device 102 to pivot and thus change an angle or orientation while being secured by the lever structure 250 of the delivery device 200, which would be desirable in advancement and/or retraction of the endovascular system in a tortuous vascular path. Alternatively, the proximal end 104p of the coupling member 104 may have other shapes such as a cylindrical or conical shape.

With reference to FIGS. 1A-1C and 2A-2C, in a specific embodiment, the distal end portion of the lever structure 250 may have features such as steps 256, forming a grasping structure or the like (FIGS. 1C and 2C). The step features 256 help contain the proximal end 104p of the coupling member 104 in the lever structure 250 when the endovascular system 100 is in the delivery configuration, as shown in FIGS. 1A-1C. When the endovascular system 100 is in a release configuration, as shown in FIGS. 2A-2C, the grasping structure 256 is opened, allowing the proximal end 104p of the coupling member 104 and thus the implantable device 102 to be released, as better viewed in FIG. 2C.

In operation, the implantable device 102 may be delivered and deployed at a target site for treatment of a disorder within the vasculature of a patient. In an embodiment for treating neurovascular conditions such as aneurysm, a microcatheter may be introduced to the target site through an access e.g., in the femoral artery or groin area of the patient by using an introducer sheath or guiding catheter. The microcatheter may be guided to the target site through the use of a guidewire. The guidewire is visible via fluoroscopy, allowing the microcatheter to be reliably advanced over the guidewire to the target site.

Once the target site has been accessed with the microcatheter tip, the guidewire can be withdrawn, clearing the lumen of the microcatheter. The endovascular system 100 including the implantable device 102 and the delivery device 200 in a delivery configuration, can be placed into the proximal open end of the microcatheter and advanced through the microcatheter. When the implantable device 102 reaches the distal end of the microcatheter, it can be deployed from the microcatheter and positioned at the target site. The physician may advance and retract the implantable device 102 several times to obtain a desirable position of the implant within the vasculature. Once the implantable device 102 is satisfactorily positioned, the physician may break or separate the distal tip section 206a of the elongate tubular member 202, and pull the detachment wire 204 to disengage it from the lever structure 250, allowing the implantable device 102 to be released at the target site. The elongate tubular member 202 can be then removed from the microcatheter, and additional implant, if necessary for proper treatment, may be delivered and deployed in the same manner. After deployment of the implantable device, the microcatheter can be withdrawn from the vasculature of the patient.

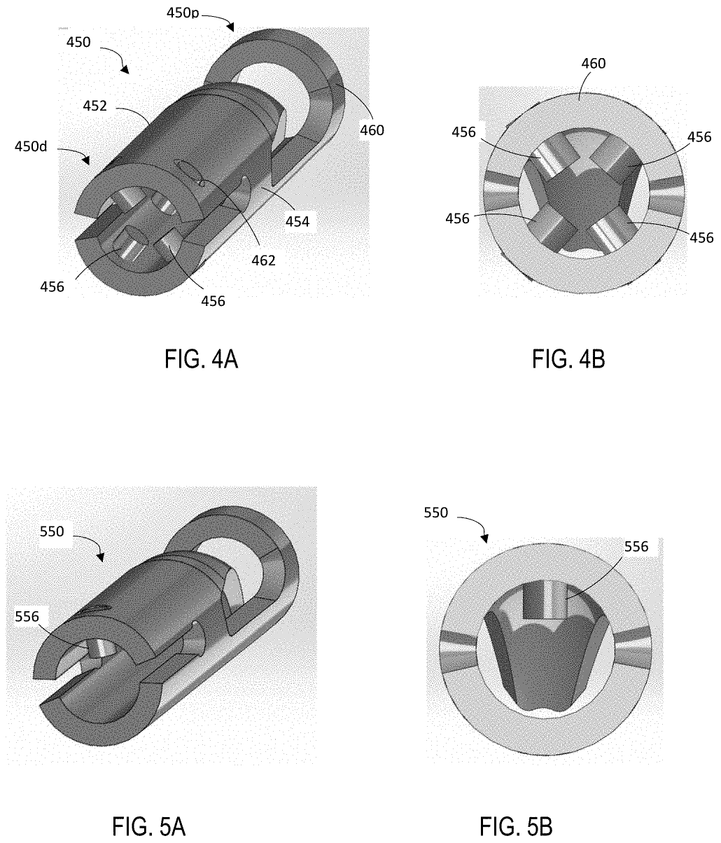

With reference to FIGS. 4A-4C through 11A-11B, alternative embodiments of a lever structure are now described. The alternative lever structures may include one or more pin members configured to retain an implantable device when in a delivery configuration. The one or more pin members may serve as a restraining gate or steps to prevent the implantable device from passing until the lever structure is actuated to open to release the implantable device.

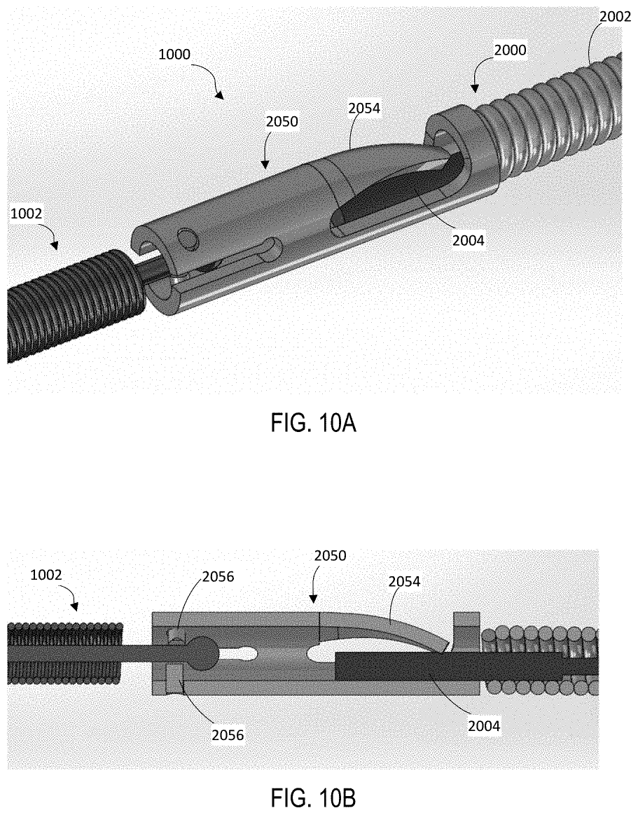

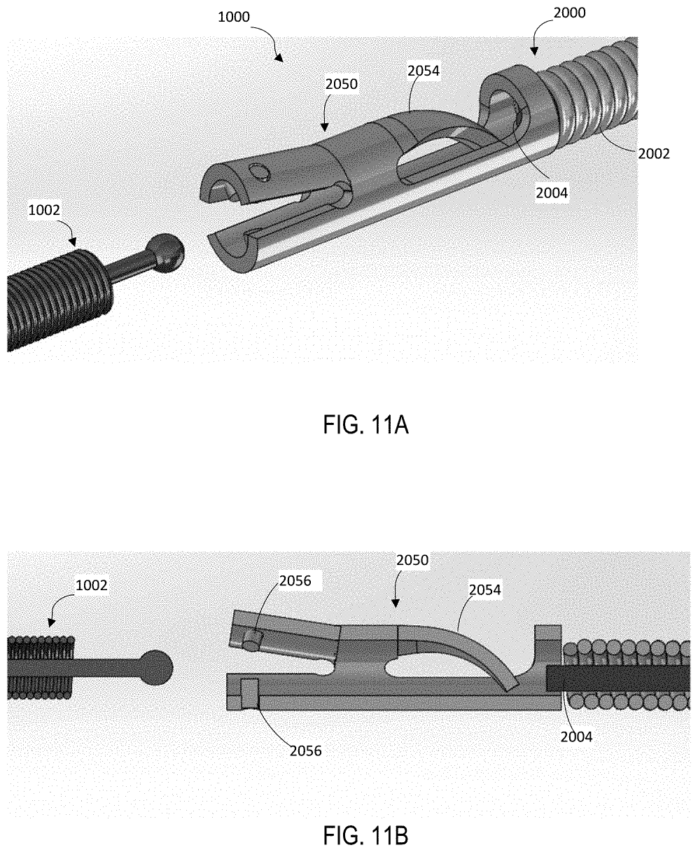

FIG. 4A is an isometric view of an example lever structure 450 according to an embodiment of the disclosure. FIG. 4B is an end view of the lever structure 450 shown in FIG. 4A. As shown, the example lever structure 450 comprises a lever member 452 pivotable about a fulcrum 454. The lever structure 450 comprises a proximal portion 450p proximal of the fulcrum 454 and a distal portion 450d distal of the fulcrum 454. At the distal portion 450d, the lever structure 450 comprises four pin members 456 protruding inwardly, forming a restraining gate or steps preventing an implantable device or a coupler affixed to the implantable device from passing when in a delivery configuration (FIGS. 10A-10B). The pin members 456 can be dimensioned or configured such that when the lever structure 450 is actuated to open its distal portion 450d, the gap between the pin members 456 is sufficiently large to allow the implantable device or the coupler affixed to the implantable device to pass to release the implantable device (FIGS. 11A-11B).

With reference to FIGS. 4A-4B, the lever structure 450 may be formed by cutting a tubular wall and affixing pinning members. By way of example, a tubular wall 460 may be cut to have a first pair of opposing cut-outs distal of a segment 454 of the tubular wall 460, a second pair of opposing cut-outs proximal of the segment 454, and an arc-shaped cut-out across the second pair of opposing cut-outs, forming the lever member 452 pivotable about the segment or fulcrum 454. The lever structure 450 can be formed such that when the lever structure 450 is in a natural state, the lever member 452 is oriented upwardly distally (e.g., FIGS. 11A-11B). This may be accomplished by using a memory material, shaping the cut-outs in the tubular wall 460, and shaping the lever member 452. In an embodiment, the proximal end portion of the lever member 452 may be shaped to have a curved outer surface and/or bent downwardly or inwardly when the lever structure 450 is in a natural state. Holes 462 may be formed in the distal portion of the tubular wall 460 and pin members 456 inserted and affixed to the holes 462 by bonding, soldering, or other suitable means.

The tubular wall 460 may be cut or shaped using laser cutting, etching, or any other suitable techniques known in the art. The tubular wall 460 can be a separate piece of component and the lever structure 450 can be formed by cutting the separate piece of component and then coupled to the distal end portion of a delivery device e.g., via soldering, welding, adhesive bonding or other suitable means. Alternatively, the tubular wall 460 can be an integral portion of an elongate tubular member of a delivery device and the lever structure 450 can be formed by cutting the tubular wall portion. The tubular wall 460 can be constructed of a material comprising nitinol, stainless steel, or other metals or metal alloys, or a biocompatible polymeric material.

FIGS. 4A-4B shows four pin members 456 at the distal portion of the lever structure 450. The four-pin or multiple-pin configuration provides greater circumferential surface area for engaging the implantable device or a coupler affixed to the implantable device, resulting in higher coupling force between the delivery device and the implantable device given a fixed pin height. It is apparent that more or fewer than four pin members can be used, and the disclosure is not limited to the number of pin members.

FIGS. 5A-5B. depict an example lever structure 550 where only one pin member 556 is provided at the distal end portion of the lever member. The height of the pin member 556 can be dimensioned to provide a sufficient contact area to engage the implantable device or a coupler affixed to the implantable device. The one-pin configuration depicted in FIGS. 5A-5B may simplify the making of the lever structure because it requires less cutting, welding or gluing in forming a hole and affixing a pin to the hole.

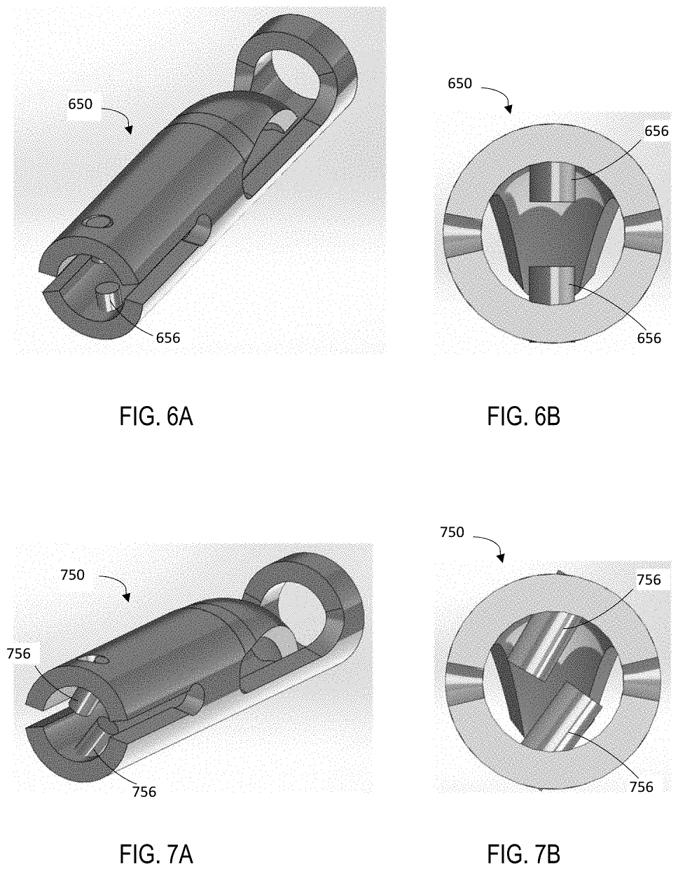

FIGS. 6A-6B. depict an example lever structure 650 where two pin members 656 are provided at the distal portion of the lever structure 650. The two pin members 656 may be positioned opposite to each other. Similar to the one-pin configuration, the two-pin configuration is relatively easy to make.

FIGS. 7A-7B. depict another example lever structure 750 where two pin members 756 are provided at the distal portion of the lever structure 750. The two pin members 756 depicted in FIGS. 7A-7B. are positioned opposite and offset relative to each other. The offset configuration can reduce the risk of ensnaring the implantable device or a coupler affixed to the implantable device on the side of the pins during release at extreme angles.

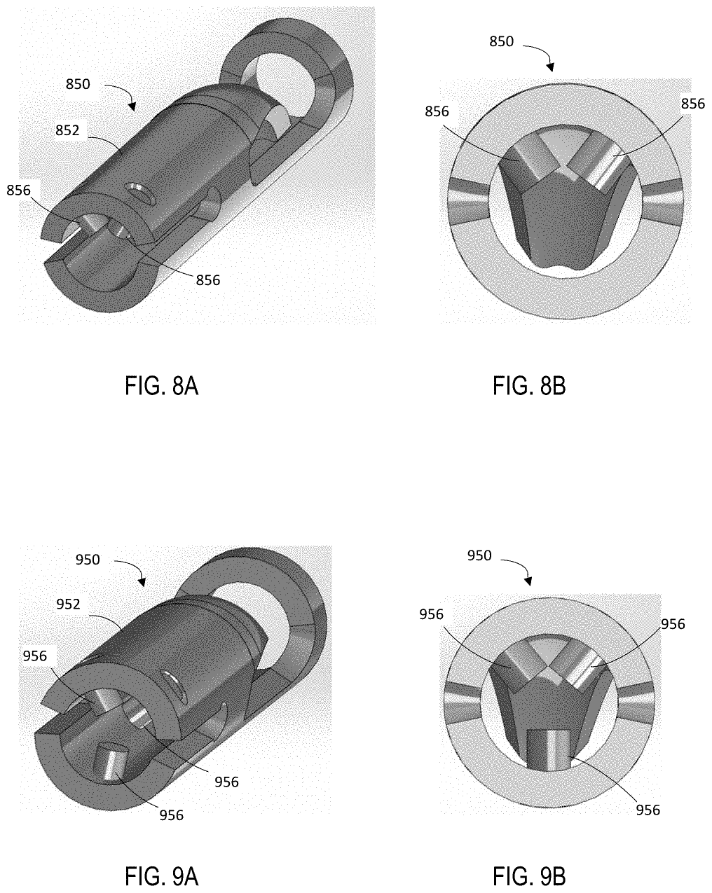

FIGS. 8A-8B. depict another example lever structure 850 where two pin members 856 are provided at the distal end portion of the lever member. Both pin members 856 are provided in the upper lever member 852 of the lever structure 850. Because the upper lever member 852 articulates upon actuation of the lever structure 850, having the pin members 856 or restraining mechanism on the upper lever member 852 can maximize the relative motion, thereby aiding in ease of release.

FIGS. 9A-9B. depict another example lever structure 950 where three pin members 956 are provided at the distal portion of the lever structure, wherein two pin members are provided in the upper lever member 952 and one pin member in the lower tubular wall or opposite to the upper two pin members. As compared with the configurations depicted in FIGS. 4A-4B through 8A-8B, the three-pin configuration depicted in FIGS. 9A-9B may provide a good balance of coupling strength, ease of manufacturing, and ease of release of the implantable device.

FIGS. 10A-10B and 11A-11B depict an example endovascular system 1000 according to embodiments of the disclosure. FIGS. 10A-10B show a delivery configuration of the endovascular system 1000. FIGS. 11A-11B show a release configuration of the endovascular system 1000. As shown, the example endovascular system 1000 includes an implantable device 1002 and a delivery device 2000 operable to deliver and deploy the implantable device 1002 at a target site in a patient. The delivery device 2000 generally includes an elongate tubular member 2002 and an elongate detachment wire 2004. A lever structure 2050 is provided at the distal end portion of the elongate tubular member 2002 to retain or release the implantable device 1002. In a delivery configuration depicted in FIGS. 10A-10B, the detachment wire 2004 is engaged with the lever member 2054 of the lever structure 2050, allowing the lever structure 2050 to retain or secure the implantable device 1002 for advancement and/or retraction in the vasculature of a patient. To deploy the implantable device 1002 at a target site, the detachment wire 2004 can be disengaged from the lever member 2054 of the lever structure 2050, allowing the lever structure 2050 to release the implantable device 1002, as depicted in FIGS. 11A-11B.

The lever structure 2050 may be any of the lever structures 450, 550, 650, 750, 850, 950 depicted in FIGS. 4A-4B through 9A-9B. The lever structure 2050 may include one or more pin members 2056 (FIGS. 10B and 11B) at the distal end portion. In a delivery configuration depicted in FIGS. 10A-10B, the detachment wire 2004 is engaged with the lever member 2054 of the the lever structure 2050, asserting an effort to the proximal portion of the lever member 2054 upwardly or outwardly and generating a load to the distal portion of the lever member 2054 downwardly or inwardly, thereby allowing the lever structure 2050 to engage the implantable device 1002. The one or more pin members 2056 may serve as a retraining gate or steps preventing the implantable device 1002 or a coupler affixed to the implantable device from passing, further securing the implantable device 1002 to the delivery device 2000. In a release configuration depicted in FIGS. 11A-11B, the detachment wire 2004 can be disengaged from the lever structure 2050, e.g., by being pulled proximally, removing the effort to the proximal portion of the lever member 2054 and the load off the distal portion of the lever member 2054, thereby allowing distal portion of the lever structure 2050 to open. The gap between the pin members 2056 is widened, allowing the implantable device 1002 to be released.

Various embodiments of an endovascular system and a detachment system for deploying implants within a human body have been described. Advantageously, the detachment system of the disclosure can enhance retainment of the implant during delivery. The concentric coupling between the implant and the delivery system significantly reduces the risks of inadvertent or premature release of the implant when the delivery system is advanced or retracted in navigating through a tortuous vascular path in the human body.

Various embodiments of an endovascular system and a detachment system for deploying implants within a human body are described with reference to figures. It should be noted that the figures are intended to facilitate illustration and some figures are not necessarily drawn to scale. Further, in the figures and description, specific details may be set forth in order to provide a thorough understanding of the disclosure. It will be apparent to one of ordinary skill in the art that some of these specific details may not be employed to practice embodiments of the disclosure. In other instances, well known components or process steps may not be shown or described in detail in order to avoid unnecessarily obscuring embodiments of the disclosure.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The terms “coupled,” “supported,” “connected,” “mounted”, and variations are used broadly and encompass both direct and indirect couplings, supports, connections, and mounting. The term “proximal” and its grammatically equivalent refers to a position, direction or orientation towards the operator or physician's side. The term “distal” and its grammatically equivalent refers to a position, direction or orientation away from the operator or physician's side.

Those skilled in the art will appreciate that various other modifications may be made. All these or other variations and modifications are contemplated by the inventors and within the scope of the invention.

What is claimed is:

1. A system for delivering and deploying an implantable device in a patient, comprising:
a tubular member having a lumen, a proximal end portion, and a distal end portion, the distal end portion of the tubular member comprising a lever structure configured to secure and release an implantable device, the lever structure comprising a fulcrum and a lever member pivotable about the fulcrum,
a detachment wire having a proximal end portion and a distal end portion extending in the lumen of the tubular member, the distal end portion of the detachment wire being configured to engage the lever structure to assert an effort to a proximal portion of the lever member outwardly, thereby generating a load to a distal portion of the lever member inwardly to allow the lever structure to engage and secure the implantable device, the distal end portion of the detachment wire being disengageable from the lever structure to remove the effort asserted to the proximal portion of the lever member, thereby removing the load off the distal portion of the lever member to allow the lever structure to disengage and release the implantable device.

2. The system of claim 1, wherein the lever member and the fulcrum are formed by cutting a tubular wall.

3. The system of claim 2, wherein the tubular wall has a first pair of opposing cut-outs distal of a segment of the tubular wall, a second pair of opposing cut-outs proximal of the segment, and an arc-shaped cut-out across the second pair of opposing cut-outs, forming the lever member pivotable about the segment.

4. The system of claim 3, wherein the lever member is formed to provide a configuration of the lever structure wherein when the lever structure is engaged with the detachment wire and the implantable device, a central longitudinal axis of the lever structure is generally concentric with a central longitudinal axis of the tubular member.

5. The system of claim 3, wherein the lever member is formed to provide a configuration of the lever structure wherein when the lever structure is disengaged from the detachment wire and the implantable device, the lever member is oriented outwardly distally.

6. The system of claim 5, wherein the distal end portion of the detachment wire has a cylindrically shaped surface dimensioned to engage an inner surface of the lever member to assert the effort to the proximal portion of the lever member outwardly.

7. The system of claim 5, wherein the distal portion of the lever structure comprises a step feature configured to facilitate retainment of the implantable device.

8. The system of claim 1, wherein the proximal end portion of the tubular member comprises a tip section that is separatable from the tubular member to allow a user to pull the detachment wire.

9. The system of claim 1, wherein the lever structure further comprises one or more pin members protruding inwardly configured to restrain the implantable device in a delivery state.

10. The system of claim 9, wherein the one or more pin members comprises at least one pin member protruding from the distal portion of the lever member.

11. The system of claim 10, wherein the one or more pin members further comprises at least one pin member opposite to the at least one pin member protruding from the distal portion of the lever member.

12. An endovascular system, comprising:
an embolic device, and
a delivery device operable to deploy the embolic device at a target site in a vasculature of a patient, wherein the delivery device comprises:
an elongate tubular member having a lumen, a proximal end portion, and a distal end portion, the distal end portion of the elongate tubular member comprising a lever structure configured to secure and release the embolic device, the lever structure comprising a fulcrum and a lever member pivotable about the fulcrum, and
an elongate detachment wire having a proximal end portion and a distal end portion extending in the lumen of the elongate tubular member, the distal end portion of the elongate detachment wire being configured to engage the lever structure to assert an effort to a proximal portion of the lever member outwardly, thereby generating a load to a distal portion of the lever member inwardly to allow the lever structure to engage and secure the embolic device, the distal end portion of the elongate detachment wire being disengageable from the lever structure to remove the effort asserted to the proximal portion of the lever member, thereby removing the load off the distal portion of the lever member to allow the lever structure to disengage and release the embolic device.

13. The endovascular system of claim 12, wherein the lever member and the fulcrum are formed by cutting a tubular wall.

14. The endovascular system of claim 13, wherein the tubular wall has a first pair of opposing cut-outs distal of a segment of the tubular wall, a second pair of opposing cut-outs proximal of the segment, and an arc-shaped cut-out across the second pair of opposing cut-outs, forming the lever member pivotable about the segment.

15. The endovascular system of claim 14, wherein the lever member is formed to provide a configuration of the lever structure wherein when the lever structure is engaged with the detachment wire and the embolic device, a central longitudinal axis of the lever structure is generally concentric with a central longitudinal axis of the elongate tubular member.

16. The endovascular system of claim 14, wherein the lever member is formed to provide a configuration of the lever structure wherein when the lever structure is disengaged with the detachment wire and the embolic device, the lever member is oriented outwardly distally.

17. The endovascular system of claim 16, wherein the distal end portion of the detachment wire has a cylindrically shaped curve surface dimensioned to engage an inner surface of the lever member to assert the effort to the proximal end portion of the lever member outwardly.

18. The endovascular system of claim 16, wherein the embolic device comprises a coupling member having a proximal end portion configured to engage an inner surface of the lever member when the load is generated to the distal portion of the lever member inwardly to secure the embolic device.

19. The endovascular system of claim 18, wherein the proximal end portion of the coupling member of the embolic device has an enlarged dimension generally in a spherical shape.

20. The endovascular system of claim 19, wherein the proximal end portion of the coupling member is pivotable when engaged with the inner surface of the lever member, thereby allowing the embolic device to pivot when secured by the lever structure.

21. The endovascular system of claim 12, wherein the distal portion of the lever structure comprises a step feature configured to facilitate retainment of the embolic device.

22. The endovascular system of claim 12, wherein the proximal end portion of the elongate tubular member comprises a tip section that is separatable from the elongate tubular member to allow a user to pull the detachment wire.

23. The endovascular system of claim 12, wherein the embolic device comprises an embolic coil, a stent, or an intrasaccular device.

24. The system of claim 12, wherein the lever structure further comprises one or more pin members protruding inwardly configured to restrain the implantable device in a delivery state.

25. The system of claim 24, wherein the one or more pin members comprises at least one pin member protruding from the distal portion of the lever member.

26. The system of claim 25, wherein the one or more pin members further comprises at least one pin member opposite to the at least one pin member protruding from the distal portion of the lever member.
